(19) United States
(12) Patent Application Publication
(10) Pub. No.: US 2004/0147027 A1
(43) Pub. Date: Jul. 29, 2004

(54) Complex for facilitating delivery of dsRNA into a cell and uses thereof

(76) Inventors: Carol M. Troy, Hastings-on-Hudson, NY (US); Lloyd A. Greene, Larchmont, NY (US)

Correspondence Address: Leslie Gladstone Restaino, Esq., Brown Raysman Millstein Felder & Steiner LLP, 163 Madison Avenue, P.O. Box 1989, Morristown, NJ, 07962-1989, US

(21) Appl. No.: 10/353,902

(22) Filed: Jan. 28, 2003

(51) Int. Cl. A61K 48/00; C12N 15/88

(52) U.S. Cl. 435/458; 514/44

(57) ABSTRACT

The present invention provides a membrane-permeable complex for facilitating the delivery of a double-stranded ribonucleic acid molecule into a cell. Specifically, the invention provides a membrane-permeable complex that comprises a double-stranded ribonucleic acid molecule, such as a small interfering RNA, a cell-penetrating peptide, and a covalent bond linking the double-stranded ribonucleic acid to the cell-penetrating peptide. Also provided are methods of using the membrane-permeable complex of the present invention to deliver the double-stranded ribonucleic acid molecule to a cell or to inhibit expression of a gene product by a cell.

### STATEMENT OF GOVERNMENT INTEREST

This invention was made with government support under NIH Grant Nos. 1 R01 NS43089 and 1 R29 NS35933. As such, the United States government has certain rights in this invention.

### BACKGROUND OF THE INVENTION

Researchers have discovered a growing number of RNAs that do not function as messenger RNAs, transfer RNAs or ribosomal RNAs. These so-called “non-coding” RNAs describe a wide variety of RNAs of incredibly diverse function, ranging from the purely structural to the purely regulatory (Riddihough, “The other RNA world,” Science, 296, 1259 (May 17, 2002)). Representative non-coding RNAs include small nuclear RNAs, involved in the splicing of pre-mRNAs in eukaryotes (Will, C. L., et al., Curr. Opin. Cell Biol., 13, 290 (2001)), small nucleolar RNAs, which direct 2′-O-ribose methylation and pseudouridylation of rRNA and tRNA (Kiss, T., EMBO J., 20, 3617 (2001)) and “micro-RNAs” (“miRNAs”), very small RNAs of approximately 22 nucleotides in length which appear to be involved in various aspects of mRNA regulation and degradation. Two miRNAs characterized in some detail are the “small temporal RNAs” (“stRNAs”) lin4 and let7, which control developmental timing in the nematode worm C. elegans and repress the translation of their target genes by binding to the 3′ untranslated regions of their mRNAs (Riddihough, “The other RNA world,” Science, 296, 1259 (May 17, 2002); Ruvkun, G., Science, 294, 797 (2001); Grosshans, H., et al., J. Cell. Biol., 156, 17 (2002)). Also known are the short hairpin RNAs (“shRNAs”), patterned from endogenously encoded triggers of the RNA interference pathway (Paddison, et al., Short hairpin RNAs (shRNAs) induce sequence specific silencing in mammalian cells. Genes and Dev., 16(8):948-958 (2002)). The non-coding RNA that has generated the most interest, however, is the “small interfering RNA” or “siRNA” associated with the phenomenon of RNA interference (“RNAi”).

Specifically, researchers have discovered that when double-stranded RNA (dsRNA) is introduced into a cell, it has the ability to silence the expression of a homologous gene within the cell, i.e., the introduced dsRNA “interferes” with gene expression. RNAi was discovered by Guo and Kemphues in 1995, when they reported that both the sense and antisense strands of test oligonuleotides disrupted the expression of par-1 in Caenorhabditis elegans, following injection into a cell (Guo, et al., “Par-1, A gene required for establishing polarity in C. elegans embryos, encodes a putative Ser/Thr kinase that is asymmetrically distributed,” Cell, 81, 611-620 (1995)). In 1998, Fire et al. clearly proved the existence and efficacy of RNAi by injecting into the gut of C. elegans a dsRNA that had been prepared in vitro (Fire, et al., “Potent and specific genetic interference by double-stranded RNA in Caenorhabditis elegans,” Nature, 391, 806-811 (1998)). The injection of dsRNA into C. elegans resulted in loss of expression of the homologous target gene, not only throughout the worm, but also in its progeny. It is now well accepted that the phenomenon of RNAi is ubiquitous among bacteria, fungi, plants, and animals, although the precise mechanism of interference may differ.

In eukaryotes, the current model of the RNAi mechanism involves both an initiation and an effector step. In the initiation step, a processing enzyme cleaves the introduced dsRNA into small interfering RNAs of 21-23 nucleotides. In the effector step, each siRNA is incorporated into an RNA induced silencing complex (“RISC”), comprising a helicase, an exonucleolytic nuclease, and an endonucleolytic nuclease. The siRNA, now incorporated into the RISC, serves as a guide molecule, directing the RISC to the homologous mRNA transcript for degradation (Hammond, S. M., et al., “Post-transcriptional gene silencing by double-stranded RNA,” Nature Rev. Gen., 2, 110-119).

RNA interference is an invaluable tool for functional genomics, since researchers can create numerous silenced phenotypes to determine the function of the targeted gene. Even greater promise may exist in the field of gene-specific therapeutics. Specifically, RNA interference offers a number of advantages over antisense technology, which is the most commonly cited approach for achieving post-transcriptional gene silencing. For example, RNAi methods are more effective and more economical than methods involving antisense nucleic acids. Since cellular uptake of unmodified antisense nucleic acid is very inefficient, a large amount of antisense nucleic acid needs to be synthesized and applied in order to achieve and maintain a sufficient concentration in the target cells—which is usually at or above the level of the endogenous target mRNA. Therefore, a successful antisense strategy requires the introduction of large amounts of single-stranded antisense nucleic acid (DNA or RNA) into cells. In contrast, the cellular uptake of double-stranded RNA is more efficient, thereby permitting RNAi to occur with much smaller amounts of dsRNA.

Even so, a need exists for improved dsRNA uptake into the cell. Previous methods of delivery known in the art primarily involve transfection (for general transfection protocols, see Elbashir, S. M., et al., “Duplexes of 21-nucleotide RNAs mediate RNA interference in mammalian cell culture,” Nature, 411, 494-498 (2001a); Elbashir, S. M., et al., “RNA interference is mediated by 21 and 22 nt RNAs,” Genes & Dev., 15, 188-200 (2001b)). The efficiency of transfection depends on cell type, passage number and the confluency of the cells. The time and the manner of formation of siRNA are also critical. Low transfection efficiencies are the most frequent cause of unsuccessful silencing.

Other techniques for dsRNA uptake include electroporation, injection, liposome-facilitated transport, and microinjection. Although direct microinjection of dsRNA into cells is generally considered to be the most effective means known for inducing RNAi, the characteristics of this technique severely limit its practical utility. In particular, direct microinjection can only be performed in vitro, which limits its application to gene therapy. Furthermore, only one cell at a time can be microinjected, which limits the technique's efficiency. As a means of introducing dsRNA into cells, electroporation is also relatively impractical because it is not possible in vivo. Finally, while dsRNA can be introduced into cells using liposome-facilitated transportation or passive uptake, these techniques are slow and inefficient.

It is also possible to introduce dsRNA indirectly into cells, by transforming the cells with expression vectors containing DNA coding for dsRNA (see, e.g., U.S. Pat. No. 6,278,039, U.S. published application 2002/0006664, WO 99/32619, WO 01/29058, WO 01/68836, and WO 01/96584). Cells transformed with the dsRNA-encoding expression vector will then produce dsRNA in vivo. While this technique is theoretically feasible, there are a number of obstacles that must be overcome before it can be widely used clinically or in industry. For example, before this strategy of RNAi is utilized for gene therapy, the following factors should be taken into consideration: availability of the expression vector, transformation efficiency of the expression vector, and vector safety.

Peptide vectors have been used to deliver various macromolecules across plasma membranes. In particular, it is known that an antisense oligonucleotide may be transported into a cell if it is conjugated to a protein/peptide vector. The protein/peptide-vector-conjugated antisense oligonucleotide will then be taken up by the cell. For example, U.S. published application 2002/0009758 discloses a means for transporting antisense nucleotides into cells using a short peptide vector, MPG. The MPG peptide contains a hydrophobic domain derived from the fusion sequence of HIV gp41, and a hydrophilic domain derived from the nuclear localization sequence of SV40 T-antigen. It has been demonstrated that several molecules of the MPG peptide coat the antisense oligonucleotide, which can then be delivered into cultured mammalian cells in less than 1 hour with relatively high efficiency (90%). Furthermore, it has been shown that the interaction with MPG strongly increases both the oligonucleotide's stability to nucleases, and its ability to cross the plasma membrane.

Similarly, U.S. Pat. No. 6,287,792 discloses a method for delivering antisense oligonucleotides to cells by first linking the oligonucleotides to biotin. The biotinylated antisense oligonucleotides then bind to avidin/avidin fusion protein, which acts as a transportation vector to assist the antisense oligonucleotides in crossing cell membranes.

U.S. Pat. No. 6,025,140 discloses the use of vector peptides to deliver antisense molecules across plasma membranes, and specifically discloses the use of penetratin and transportan to transport peptide nucleic acids across cell membranes.

Accordingly, the so called “cell-penetrating peptides” offer certain advantages for protocols involving the translocation of macromolecules into cells, including non-traumatic internalization, limited endosomal degradation, high translocation efficiencies at low concentrations, and delivery to a wide variety of cell types.

However, none of the above-noted references disclose the use of vector peptides to transport double-stranded ribonucleic acids across cell membranes for the purpose of RNA interference.

While there are various methods available for directly and indirectly introducing dsRNA into cells, it is clear that these methods are generally inefficient, and have practical limitation. Therefore, in view of the foregoing, there exists a need to develop tools and methods for the more efficient introduction of dsRNA into cells for the purpose of achieving RNAi.

### SUMMARY OF THE INVENTION

The present invention provides a membrane-permeable complex for facilitating the delivery of a double-stranded ribonucleic acid molecule into a cell, as well as various uses of the complex. Specifically, the membrane-permeable complex described herein comprises a double-stranded ribonucleic acid molecule, a cell-penetrating peptide, and a covalent bond linking the double-stranded ribonucleic acid molecule to the cell-penetrating peptide. The present invention allows for the introduction of a double-stranded RNA molecule, such as a small interfering RNA, into a cell with greater ease and efficiency than previously possible using conventional methods known in the art, such as transfection, electroporation, liposomal delivery or microinjection. Further, use of the membrane-permeable complex of the present invention avoids many of the safety, availability and efficacy concerns of using a dsRNA expression vector to mediate delivery of double-stranded ribonucleic acid into a cell. Accordingly, the membrane-permeable complex of the present invention provides a powerful tool for various therapeutic and research applications requiring the delivery of dsRNA into a cell.

The present invention further provides various methods of using the membrane-permeable complex described herein, including a method of facilitating delivery of a double-stranded ribonucleic acid molecule into a cell. As described herein, a membrane-permeable complex comprising (a) a double-stranded ribonucleic acid molecule, (b) a cell-penetrating peptide, and (c) a covalent bond linking the double-stranded ribonucleic acid molecule to the cell-penetrating peptide, is contacted with the cell, thereby resulting in delivery of the double-stranded ribonucleic acid molecule into the cell.

Finally, the present invention discloses a method of determining the function of a target gene in a cell. First, a membrane-permeable complex for inhibiting expression of the target gene is contacted with the cell, wherein the membrane-permeable complex comprises (i) a double-stranded ribonucleic acid molecule, with at least one strand of said molecule having a nucleotide sequence which is homologous to a portion of mRNA transcribed from the target gene, (ii) a cell-penetrating peptide, and (iii) a covalent bond linking the double-stranded ribonucleic acid molecule to the cell-penetrating peptide. Once the complex is delivered into the cell in an amount sufficient to inhibit expression of the target gene, the phenotype of the contacted cell is compared to that of an appropriate control cell, thereby allowing for the determination of information regarding the function of the target gene in the cell.

Additional aspects of the present invention will be apparent in view of the detailed description, which follows.

### BRIEF DESCRIPTION OF THE FIGURES

FIG. 1 illustrates that vector-linked small interference RNA (siRNA) is taken up rapidly by neurons. Sympathetic neurons were isolated from newborn mice, and grown on coverglass chamber slides for 5 days. Cultures were then treated with 80 nM V-Casp8-FITC-siRNA. Cells were examined within 10 min by confocal microscopy, to detect uptake of FITC-labeled siRNA.

FIG. 2 shows that vector-linked siRNA remains in the neurons for at least two days. Hippocampal neurons were isolated from E18 embryos, and grown on coverglass chamber slides for 5 days. Cultures were then treated with 80 nM V-Casp8-FITC-siRNA. Cells were examined two days after treatment, by confocal microscopy, to detect the presence of FITC-labeled siRNA.

FIGS. 3A and 3B illustrate that vector-linked siRNA targeted to caspase-8 inhibits expression of caspase-8 in sympathetic neurons. Sympathetic neurons were isolated from newborn mice, and grown on coverglass chamber slides for 5 days. Cultures were then treated with 80 nM V-Casp8-siRNA for one day, fixed and double-labeled with anti-caspase-8 (green) and Hoechst nuclear stain (blue), and examined with fluorescence microscopy. Caspase-8 activity can be seen in the control culture. Anti-caspase-8 activity is depicted by arrows (3A). No caspase-8 activity is seen in the culture treated with V-Casp8-siRNA—only nuclear staining is seen (3B).

FIGS. 4A and 4B show that vector-linked siRNA targeted to caspase-9 inhibits expression of caspase-9 in sympathetic neurons. Sympathetic neurons were isolated from newborn mice, and grown on coverglass chamber slides for 5 days. Cultures were then treated with 40 nM V-Casp9-siRNA for one day, fixed and double-labeled with anti-caspase-9 (green) and Hoechst nuclear stain (blue), and examined with fluorescence microscopy. Caspase-9 activity can be seen in the control culture. Anti-caspase-9 activity is depicted by arrows (4A). No caspase-9 activity is seen in the culture treated with V-Casp9-siRNA—only nuclear staining is seen (4B).

FIG. 5 illustrates that vector-linked siRNA targeted to SOD1 inhibits SOD1-specific activity. Hippocampal neurons were isolated from E18 embryos, and grown in culture for 5 days. Cells were then treated with various concentrations of V-SOD1i (siRNA targeted to SOD1). After 4 h, cells were harvested and assayed for SOD activity.

FIG. 6 shows that vector-linked siRNA targeted to SOD1 is more effective than vector-linked antisense oligonucleotide. Hippocampal neurons were isolated from E18 embryos, and grown in culture for 5 days. Cells were then treated with various concentrations of either V-SOD1i (siRNA targeted to SOD1) or V-ASOD1 (antisense oligonucleotide targeted to SOD1). After one day of treatment, relative neuronal survival was determined.

FIG. 7 shows that the vector can be linked to the sense or antisense strand of siRNA. Hippocampal neurons were isolated from E18 embryos, and grown in culture for 5 days. Cells were then treated with SOD1-siRNA linked to the sense or antisense strand. After one day of treatment, relative neuronal survival was determined.

### DETAILED DESCRIPTION OF THE INVENTION

The present invention describes a membrane-permeable complex for facilitating the delivery of a double-stranded ribonucleic acid molecule into a cell, as well as various uses of the complex. Specifically, it has been found that a cell-penetrating peptide may be covalently bonded to a double-stranded ribonucleic acid molecule to form a membrane-permeable complex. Advantageously, the use of the complex yields an unprecedented and unexpected 100% transfection efficiency of dsRNA into neuronal cells. Such unprecedented uptake efficiency allows for the efficient in vivo delivery of dsRNA into tissues, and by extension, into entire organisms, thereby expanding the therapeutic possibilities of RNA interference applications. While the present invention is primarily directed to the delivery of a double-stranded ribonucleic acid molecule into a cell for the purposes of RNA interference, the membrane-permeable complex described herein may also be used to facilitate the delivery of other non-coding RNAs, such as small temporal RNAs, small nuclear RNAs, small nucleolar RNAs or microRNAs, which may be used in applications other than RNA interference.

The membrane-permeable complex described herein comprises a double-stranded ribonucleic acid molecule, a cell-penetrating peptide, and a covalent bond linking the double-stranded ribonucleic acid molecule to the cell-penetrating peptide.

As used herein, a “double-stranded ribonucleic acid molecule” refers to any RNA molecule, fragment or segment containing two strands forming an RNA duplex, notwithstanding the presence of single stranded overhangs of unpaired nucleotides. Further, as used herein, a double-stranded ribonucleic acid molecule includes single stranded RNA molecules forming functional stem-loop structures, such as small temporal RNAs, short hairpin RNAs and microRNAs, thereby forming the structural equivalent of an RNA duplex with single strand overhangs. The RNA molecule of the present invention may be isolated, purified, native or recombinant, and may be modified by the addition, deletion, substitution and/or alteration of one or more nucleotides, including non-naturally occurring nucleotides, including those added at 5′ and/or 3′ ends to increase nuclease resistance.

The double-stranded ribonucleic acid molecule of the membrane-permeable complex may be any one of a number of non-coding RNAs (i.e., RNA which is not mRNA, tRNA or rRNA), including, preferably, a small interfering RNA, but may also comprise a small temporal RNA, small nuclear RNA, small nucleolar RNA, short hairpin RNA or a microRNA having either a double-stranded structure or a stem loop configuration comprising an RNA duplex with or without single strand overhangs. The double-stranded RNA molecule may be very large, comprising thousands of nucleotides, or preferably in the case of RNAi protocols involving mammalian cells, may be small, in the range of 21-25 nucleotides. Accordingly, a “small interfering RNA”, as used herein, refers to a double stranded RNA duplex of any length, with or without single strand overhangs, wherein at least one strand, putatively the antisense strand, is homologous to the target mRNA to be degraded. In a preferred embodiment, the siRNA of the present invention comprises a double-stranded RNA duplex of at least 19 nucleotides, and even more preferably, comprises a 21 nucleotide sense and a 21 nucleotide antisense strand paired so as to have a 19 nucleotide duplex region and a 2 nucleotide overhang at each of the 5′ and 3′ ends. Even more preferably, the 2 nucleotide 3′ overhang comprises 2′ deoxynucleotides, e.g., TT, for improved nuclease resistance.

In a preferred embodiment of the invention, at least one strand of the double-stranded ribonucleic acid molecule (i.e., the antisense strand) of the membrane-permeable complex is homologous to a portion of mRNA transcribed from the SOD1 gene, preferably the human SOD 1 gene. More preferably, the double-stranded ribonucleic acid is a small interfering RNA targeted to the nucleotide sequence of SEQ ID NO: 1 or SEQ ID NO: 2. As used herein, “homologous” refers to a nucleotide sequence that has at least 80% sequence identity, preferably at least 90% sequence identity, more preferably at least 95% sequence identity, and even more preferably at least 98% sequence identity, to a portion of mRNA transcribed from the target gene. Specifically, the small interfering RNA must be of sufficient homology to guide the RISC to the target mRNA for degradation. Limited mutations in siRNA relative to the target mRNA reduces, but does not entirely abolish, target mRNA. Accordingly, the most preferred embodiment of the invention comprises a siRNA having 100% sequence identity with the target mRNA.

In another embodiment, at least one strand of the double-stranded ribonucleic acid molecule of the membrane-permeable complex is homologous to a portion of mRNA transcribed from the caspase 8 gene, preferably the human caspase 8 gene. Preferably, the double-stranded ribonucleic acid molecule is a small interfering RNA comprising the nucleotide sequence of SEQ ID NO: 3 or SEQ ID NO: 4. In yet another embodiment, at least one strand of the double-stranded ribonucleic acid molecule of the membrane-permeable complex is homologous to a portion of mRNA transcribed from the caspase 9 gene, preferably human caspase 9. Preferably, the double-stranded ribonucleic acid molecule is a small interfering RNA comprising the nucleotide sequence of SEQ ID NO: 5.

In the practice of the present invention, at least one strand of the double-stranded ribonucleic acid molecule (either the sense or the antisense strand) is to be modified for linkage with a cell-penetrating peptide, for example, with a thiol group, so that the covalent bond links the modified strand to the cell-penetrating peptide. Where the strand is modified with a thiol group, the covalent bond linking the cell-penetrating peptide and the modified strand of the ribonucleic acid molecule can be a disulfide bond, as is the case where the cell-penetrating peptide has a free thiol function (i.e., pyridyl disulfide or a free cysteine residue) for coupling. However, it will be apparent to those skilled in the art that a wide variety of functional groups may be used in the modification of the ribonucleic acid, so that a wide variety of covalent bonds may be applicable, including, but not limited to, ester bonds, carbamate bonds and sulfonate bonds.

In a preferred embodiment of the invention, it is the 5′ end of at least one strand of the double-stranded ribonucleic acid that is modified for linkage with the cell-penetrating peptide, for instance, with a group having a thiol function (e.g., a 5′ amino-C6 linker), thereby leaving the 3′ OH end of the strand free. Alternatively, where activity of the double-stranded ribonucleic acid molecule is not adversely affected (i.e., there is no significant reduction in degradation of target mRNA), at least one strand of the double-stranded ribonucleic acid may be modified at its 3′ end for linkage with the cell-penetrating peptide, where the covalent bond links the 3′ modified strand to the cell-penetrating peptide (Holen, T., et al., “Positional effects of short interfering RNAs targeting the human coagulation trigger Tissue Factor,” Nucleic Acids Res., 30(8), 1757-1766 (Apr. 15, 2002)).

A label may also be affixed to at least one strand of the double-stranded ribonucleic acid molecule, including an enzyme label, a chemical label, or a radioactive label. Common enzymatic labels include horseradish peroxidase, biotin/avidin/streptavidin labeling, alkaline phosphatase and beta-galactosidase. Chemical labels include fluorescent agents, such as fluorescein and rhodamine, fluorescent proteins, such as phycocyanin or green fluorescent protein, and chemiluminescent labels. Fluorescein may be linked to the ribonucleic acid by using the reactive derivative fluorescein isothiocyanate (FITC). Finally, common radioactive labels include ³H, ¹³¹I and ⁹⁹Tc. Again, in a preferred embodiment, the label is affixed to the 5′ end of the strand, although the label may be attached at the 3′ end of the strand where such attachment does not significantly affect the activity of the double-stranded ribonucleic acid molecule.

The membrane-permeable complex described herein comprises a cell-penetrating peptide covalently bonded to the double-stranded ribonucleic acid molecule. Several features make cell-penetrating peptides unique vehicles for transporting biologically important molecules into cells. In particular, the activity of cell-penetrating peptides is generally non-cell-type specific. Additionally, cell-penetrating peptides typically function with high efficiency, even at low concentrations. Furthermore, the penetration of cell-penetrating peptides through cell membranes is often independent of endocytosis, energy requirements, receptor molecules, and transporter molecules. Thus, cell-penetrating peptides can efficiently deliver large cargo molecules into a wide variety of target cells (Derossi, et al., “Trojan peptides: the penetratin system for intracellular delivery,” Trends Cell Biol., 8(2), 84-87 (February 1998); Dunican, et al., “Designing cell-permeant phosphopeptides to modulate intracellular signaling pathways,” Biopolymers, 60(1), 45-60 (2001); Hallbrink, et al., “Cargo delivery kinetics of cell-penetrating peptides,” Biochim. Biophys. Acta, 1515(2), 101-109 (Dec. 1, 2001); Bolton, et al., “Cellular uptake and spread of the cell-permeable peptide penetratin in adult rat brain,” Eur. J. Neurosci., 12(8), 2847-2855 (August 2000); Kilk, et al., “Cellular internalization of a cargo complex with a novel peptide derived from the third helix of the islet-1 homeodomain. Comparison with the penetratin peptide,” Bioconjug. Chem., 12(6), 911-916 (November-December 2001)).

As used herein, a “cell-penetrating peptide” is a peptide that comprises a short (about 12-30 residues) amino acid sequence or functional motif that confers the energy-independent (i.e., non-endocytotic) translocation properties associated with the transport of the membrane-permeable complex across the plasma and/or nuclear membranes of a cell. The cell-penetrating peptide used in the membrane-permeable complex of the present invention preferably comprises at least one non-functional cysteine residue free or derivatized to form a disulfide link with a double-stranded ribonucleic acid which has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS and MAP.

In the most preferred embodiment, the cell-penetrating peptide of the membrane-permeable complex is penetratin, comprising the peptide sequence RQIKIWFQNRRMKWKK (SEQ ID NO: 6) and conservative variants thereof. As used herein, a “conservative variant” is a peptide having one or more amino acid substitutions, wherein the substitutions do not adversely affect the shape, and therefore, the biological activity (i.e., transport activity) or membrane toxicity of the cell-penetrating peptide. Penetratin is a 16-amino-acid polypeptide derived from the third alpha-helix of the homeodomain of Drosophila antennapedia. Its structure and function have been well studied and characterized (see, e.g., Derossi, et al., “Trojan peptides: the penetratin system for intracellular delivery,” Trends Cell Biol., 8(2), 84-87 (February 1998); Dunican, et al., supra; Hallbrink, et al., supra; Bolton, et al., supra; Kilk, et al., supra; Bellet-Amalric, et al., “Interaction of the third helix of Antennapedia homeodomain and a phospholipid monolayer, studied by ellipsometry and PM-IRRAS at the air-water interface,” Biochim. Biophys. Acta, 1467(1), 131-143 (Jul. 31, 2000); Fischer, et al., “Structure-activity relationship of truncated and substituted analogues of the intracellular delivery vector. Penetratin,” J. Pept. Res., 55(2), 163-172 (February 2000); Thoren, et al., “The antennapedia peptide penetratin translocates across lipid bilayers—the first direct observation,” FEBS Lett., 482(3), 265-268 (Oct. 6, 2000)). It has been shown that penetratin efficiently carries avidin, a 63-kDa protein, into human Bowes melanoma cells (Kilk, et al., supra). Additionally, it has been shown that the transportation of penetratin and its cargo is non-endocytotic and energy-independent, and does not depend upon receptor molecules or transporter molecules. Furthermore, it is known that penetratin is able to cross a pure lipid bilayer (Thoren, et al., supra). This feature enables penetratin to transport its cargo, free from the limitation of cell-surface receptor/transporter availability. The delivery vector has been shown previously to enter all cell types (Derossi, et al., supra), and effectively deliver peptides (Troy, et al., “The contrasting roles of ICE family proteases and interleukin-1beta in apoptosis induced by trophic factor withdrawal and by copper/zinc superoxide dismutase down-regulation,” Proc. Natl. Acad. Sci. USA, 93, 5635-5640 (1996)) or antisense oligonucleotides (Troy, et al., “Downregulation of Cu/Zn superoxide dismutase leads to cell death via the nitric oxide-peroxynitrite pathway,” J. Neurosci., 16, 253-261 (1996); Troy, et al., “Nedd2 is required for apoptosis after trophic factor withdrawal, but not superoxide dismutase (SOD1) downregulation, in sympathetic neurons and PC12 cells,” J. Neurosci., 17, 1911-1918 (1997)).

Other cell-penetrating peptides that may be used include transportan, pISl, Tat(48-60), pVEC, MAP and MTS. Transportan is a 27 amino acid long peptide containing 12 functional amino acids from the amino terminus of the neuropeptide galanin and mastoparan in the carboxyl terminus, connected by a lysine (Pooga, M., et al., “Cell penetration by transportan,” FASEB J., 12(1), 67-77 (1998)). It comprises the amino acid sequence GWTLNSAGYLLGKINLKALAALAKKIL (SEQ ID NO: 7) and conservative variants thereof.

pIsl is derived from the third helix of the homeodomain of the rat insulin 1 gene enhancer protein (Magzoub, et al., “Interaction and structure induction of cell-penetrating peptides in the presence of phospholipid vesicles,” Biochim. Biophys. Acta, 1512(1), 77-89 (May 2, 2001); Kilk, et al., supra), and comprises the amino acid sequence PVIRVWFQNKRCKDKK (SEQ ID NO: 8) and conservative variants thereof.

Tat is a transcription activating factor of 86-102 amino acids that allows translocation across the plasma membrane of an HIV infected cell to transactivate the viral genome (Hallbrink, M., et al., “Cargo delivery kinetics of cell-penetrating peptides,” Biochim Biophys Acta, 1515(2), 101-109 (2001); Suzuki, T., et al., “Possible Existence of Common Internalization Mechanisms among Arginine-rich Peptides,” J. Biol. Chem., 277(4), 2437-2443 (2002); Futaki, S., et al., “Arginine-rich peptides. An abundant source of membrane-permeable peptides having potential as carriers for intracellular protein delivery,” J. Biol. Chem., 276(8), 5836-5840 (2001)). A small Tat fragment extending from residues 48-60 has been determined to be responsible for nuclear import (Vives, et al., “A truncated HIV-1 Tat Protein Basic Domain rapidly translocates through the plasma membrane and accumulates in the cell nucleus,” J. Biol. Chem., 272(25), 16010-16017 (1997)) and comprises the amino acid sequence GRKKRRQRRRPPQ (SEQ ID NO: 9) and conservative variants thereof.

pVEC is an 18 amino acid long peptide derived from the murine sequence of the cell adhesion molecule vascular endothelial cadherin, extending from amino acid 615-632 (Elmquist, A., et al., “VE-cadherin-derived cell-penetrating peptide, pVEC, with carrier functions,” Exp. Cell Res., 269(2), 237-244 (2001)), and comprises the amino acid sequence LLIILRRRIRKQAHAH (SEQ ID NO: 10) and conservative variants thereof.

MTS or membrane translocating sequences are those portions of certain peptides which are recognized by acceptor proteins responsible for directing nascent translation products into the appropriate cellular organelles for further processing (Lindgren, M., et al., “Cell-penetrating peptides,” Trends in Pharmacological Sciences, 21(3), 99-103 (2000); Brodsky, J. L., “Translocation of proteins across the endoplasmic reticulum membrane,” Int. Rev. Cyt., 178, 277-328 (1998); Zhao Y, et al., “Chemical engineering of cell penetrating antibodies,” J. Immunol. Methods, 254(1-2), 137-145 (2001)). An MTS of particular relevance is MPS peptide, a chimera of the hydrophobic terminal domain of the viral gp41 protein and the nuclear localization signal from simian virus 40 large antigen, which is one combination of nuclear localization signals and membrane translocation sequences that has been shown to internalize independent of temperature and function as a carrier for oligonucleotides (Lindgren, M., et al., “Cell-penetrating peptides. Trends in Pharmacological Sciences, 21(3), 99-103 (2000); Morris, M. C., et al., “A new peptide vector for efficient delivery of oligonucleotides into mammalian cells,” Nucleic Acids Res., 25, 2730-2736 (1997)). MPS comprises the amino acid sequence GALFLGWLGAAGSTMGAWSQPKKKRKV (SEQ ID NO: 11) and conservative variants thereof.

MAPs, or model amphipathic peptides, are a group of peptides having as their essential feature helical amphipathicity and a length of at least four complete helical turns. (Scheller, et al., “Structural requirements for cellular uptake of alpha-helical amphipathic peptides,” J. Peptide Science, 5(4), 185-194 (April 1999); Hallbrink, M., et al., “Cargo delivery kinetics of cell-penetrating peptides,” Biochim Biophys Acta, 1515(2), 101-109 (Dec. 1, 2001)). An exemplary model amphipathic peptide comprises the amino acid sequence KLALKLALKALKAALKLA-amide (SEQ ID NO: 12) and conservative variants thereof.

The cell-penetrating peptides and the double-stranded ribonucleic acids described above are covalently bonded to form the membrane-permeable complex of the present invention. The general strategy for conjugation is to prepare the cell-penetrating peptide and double-stranded ribonucleic acid components separately, each modified or derivatized with appropriate reactive groups to allow for linkage between the two. The modified double-stranded ribonucleic acid is then incubated together with a cell-penetrating peptide that is prepared for linkage, for a sufficient time and under such appropriate conditions of temperature, pH, molar ratio, etc., so as to generate a covalent bond between the cell-penetrating peptide and the double-stranded ribonucleic acid molecule. Numerous methods and strategies of conjugation will be readily apparent to one of ordinary skill in the art, as will the conditions required for efficient conjugation. By way of example only, one such strategy for conjugation is as follows. In order to generate a disulfide bond between the double-stranded ribonucleic acid molecule and the cell-penetrating peptide, the 3′ or 5′ end of the dsRNA molecule is modified with a thiol group and a nitropyridyl leaving group is manufactured on a cysteine residue of the cell-penetrating peptide. However, any suitable bond may be manufactured according to methods generally and well known in the art (e.g., thioester bonds, thioether bonds, carbamate bonds, etc.). Both the derivatized or modified cell-penetrating peptide and the modified double-stranded ribonucleic acid are reconstituted in RNase/DNase sterile water, and then added to each other in amounts appropriate for conjugation, e.g., equimolar amounts. The conjugation mixture is then incubated for 15 minutes at 65° C., followed by 60 minutes at 37° C., and then stored at 4° C. Linkage can be checked by running the vector-linked siRNA and an aliquot that has been reduced with DTT on a 15% non-denaturing PAGE. siRNA can then be visualized with SyBrGreen.

The present invention further provides various methods of using the membrane-permeable complex described herein. To wit, a method of facilitating delivery of a double-stranded ribonucleic acid molecule into a cell is disclosed. In the disclosed method, a membrane-permeable complex comprising (a) a double-stranded ribonucleic acid molecule, (b) a cell-penetrating peptide, and (c) a covalent bond linking the double-stranded ribonucleic acid molecule to the cell-penetrating peptide, is contacted with the cell, thereby resulting in delivery of the double-stranded ribonucleic acid molecule into the cell. The membrane-permeable complex is contacted with the cell under such conditions of concentration, temperature and pH, etc., and for a sufficient time, to result in delivery of the complex into the cell. Specific protocols using the membrane-permeable complex of the present invention will vary according to cell type, passage number, cell-penetrating peptide used, etc., but will be readily apparent to one of ordinary skill in the art.

In a preferred embodiment, at least one strand of the double-stranded ribonucleic acid molecule is modified at its 5′ end for linkage with the cell-penetrating peptide, and the covalent bond links the 5′ modified strand to the cell-penetrating peptide. The 5′ end may be modified with a group having a thiol function, and the covalent bond linking the modified 5′ end with the cell-penetrating peptide may be a disulfide bond, such as would be the case where the cell-penetrating peptide has a free thiol group or group of corresponding function for attachment. Alternatively, where function of the double-stranded ribonucleic acid molecule is not adversely affected by such modification, at least one strand of the double-stranded ribonucleic acid molecule may be modified at its 3′ end for linkage with the cell-penetrating peptide, where the covalent bond links the 3′ modified strand to the cell-penetrating peptide. In a preferred embodiment of the present invention, the double-stranded ribonucleic acid molecule is a small interfering RNA, although other embodiments of the disclosed method contemplate the use of other non-coding RNAs, including small temporal RNAs, small nuclear RNAs, small nucleolar RNAs, and microRNAs.

Where the membrane-permeable complex of the present invention is delivered to a cell for the purposes of inhibiting expression of a target gene within the cell, i.e., for RNA interference, the double-stranded ribonucleic acid molecule delivered as part of the membrane-permeable complex is preferably a small interfering RNA. Further, at least one strand of the small interfering RNA is homologous to a portion of mRNA transcribed from the target gene. In a preferred embodiment, the siRNA strand is at least 85% homologous to a portion of mRNA transcribed from the target gene. Preferably, the siRNA strand is 90% homologous, more preferably is 95% homologous, and even more preferably, is 98% homologous to a portion of mRNA transcribed from the target gene. In the most preferred embodiment, at least one strand of the siRNA is 100% homologous to a portion of mRNA transcribed from the target gene.

The target gene may be an endogenous gene in relation to the cell, as in the case of a regulatory gene or a gene coding for a native protein, or it may be heterologous in relation to the cell, as in the case of a viral or bacterial gene, transposon, or transgene. In either case, uninhibited expression of the target gene may result in a disease or a condition. The cell is contacted with the membrane-permeable complex so that the complex is delivered into the cell in an amount sufficient to inhibit expression of the target gene.

The cell receiving the membrane-permeable complex of the present invention may be isolated, within a tissue, or within an organism. It may be an animal cell, a plant cell, a fungal cell, a protozoan, or a bacterium. An animal cell may be derived from vertebrates or invertebrates, but in a preferred embodiment of the invention, the cell is derived from a mammal, such as a rodent or a primate, and even more preferably, is derived from a human. The cell may be of any type, including epithelial cells, endothelial cells, muscle cells or nerve cells. Representative cell types include, but are not limited to, myoblasts, fibroblasts, astrocytes, neurons, oligodendrocytes, macrophages, myotubes, lymphocytes, NIH3T3 cells, PC12 cells, and neuroblastoma cells. Such delivery may be accomplished either in vitro or in vivo.

Where delivery is made in vivo to a living organism, administration may be by any procedure known in the art, including but not limited to, oral, parenteral, rectal, intradermal, transdermal or topical administration. To facilitate delivery, the membrane-permeable complex of the present invention may be formulated in various compositions with a pharmaceutically acceptable carrier, excipient or diluent. “Pharmaceutically acceptable” means the carrier, excipient or diluent of choice does not adversely affect the biological activity of the membrane-permeable complex, or the recipient of the composition.

Suitable pharmaceutical carriers, excipients and/or diluents include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water.

For oral administration, the composition may be presented as capsules or tablets, powders, granules or a suspension. The composition may be further presented in convenient unit dosage form, and may be prepared using a controlled-release formulation, buffering agents and/or enteric coatings.

For parenteral administration (i.e., subcutaneous, intravenous, or intramuscular administration), the membrane-permeable complex may be dissolved or suspended in a sterile aqueous or non-aqueous isotonic solution, containing one or more of the carriers, excipients or diluents noted above. Such formulations may be prepared by dissolving a composition containing the membrane-permeable complex in sterile water containing physiologically compatible substances such as sodium chloride, glycine, and the like, and having a buffered pH compatible with physiological conditions to produce an aqueous solution. Alternatively, a composition containing the membrane-permeable complex may be dissolved in non-aqueous isotonic solutions of polyethylene glycol, propylene glycol, ethanol, corn oil, cottonseed oil, peanut oil, etc.

The membrane-permeable complex may be administered rectally by formulation with any suitable carrier that is solid at room temperature but dissolves at body temperature. Such carriers include cocoa butter, synthetic mono-, di-, or tri-glycerides, fatty acids, polyethylene glycols, glycerinated gelatin, hydrogenated vegetable oils, and the like.

Intradermal administration of the membrane-permeable complex, i.e., administration via injectable preparation, may be accomplished by suspending or dissolving the membrane-permeable complex in a non-toxic parenterally acceptable diluent or solvent, e.g., as a solution in 1,3-butanediol, water, Ringer's solution, and isotonic sodium chloride solution. Occasionally, sterile fixed oils or fatty acids are employed as a solvent or suspending medium.

For transdermal or topical administration, the membrane-permeable complex may be combined with compounds that act to increase the permeability of the skin and allow passage of the membrane-permeable complex into the bloodstream. Such enhancers include propylene glycol, polyethylene glycol, isopropanol, ethanol, oleic acid, N-methylpyrrolidone, and the like. Delivery of such compositions may be via transdermal patch or iontophoresis device.

Specific formulations of compounds for therapeutic treatment are discussed in Hoover, J. E., Remington's Pharmaceutical Sciences (Easton, Pa.: Mack Publishing Co., 1975) and Liberman, H. A., and Lachman, L., Eds., Pharmaceutical Dosage Forms (New York, N.Y.: Marcel Decker Publishers, 1980).

The quantity of membrane-permeable complex administered to tissue or to a subject should be an amount that is effective to inhibit expression of the target gene within the tissue or subject, and are readily determined by the practitioner skilled in the art. Specific dosage will depend further upon the siRNA used, the target gene to be inhibited and the cell type having target gene expression. Quantities will be adjusted for the body weight of the subject and the particular disease or condition being targeted.

Finally, the present invention discloses a method of determining the function of a target gene in a cell. First, a membrane-permeable complex for inhibiting expression of the target gene is contacted with the cell, wherein the membrane-permeable complex comprises (i) a double-stranded ribonucleic acid molecule, with at least one strand of said molecule having a nucleotide sequence which is homologous to a portion of mRNA transcribed from the target gene, (ii) a cell-penetrating peptide, and (iii) a covalent bond linking the double-stranded ribonucleic acid molecule to the cell-penetrating peptide. Once the complex is delivered into the cell in an amount sufficient to inhibit expression of the target gene, the phenotype of the contacted cell is compared to that of an appropriate control cell, thereby allowing for the determination of information regarding the function of the target gene in the cell.

The present invention is described in the following examples, which are set forth to aid in the understanding of the invention, and should not be construed to limit in any way the scope of the invention as defined in the claims which follow thereafter.

### EXAMPLES

### Example 1

Targets for siRNA were designed for various mRNAs. A general strategy for designing siRNA targets comprises beginning with an AUG stop codon and then scanning the length of the desired cDNA for AA dinucleotide sequences. The 3′ 19 nucleotides adjacent to the AA sequences are recorded as potential siRNA target sites. The potential target site can then be compared to the appropriate genome database, so that any target sequences that have significant homology to non-target genes can be discarded. Multiple target sequences along the length of the gene should be located, so that target sequences are derived from the 3′, 5′ and medial portions of the mRNA. Negative control siRNAs can be generated using the same nucleotide composition as the subject siRNA, but scrambled and checked so as to lack sequence homology to any genes of the cells being transfected. (Elbashir, S. M., et al., “Duplexes of 21-nucleotide RNAs mediate RNA interference in cultured mammalian cells,” Nature, 411, 494-498 (2001); Ambion siRNA Design Protocol, at www.ambion.com).

In the present case, generated target sequences were 21 bases long, beginning with AA, and modified with a thiol group at the 5′ C6 carbon on one strand. Custom siRNAs were generated on order from Dharmacon Research, Inc., Lafayette, Colo. Other sources for custom siRNA preparation include Xeragon Oligonucleotides, Huntsville, Ala. and Ambion of Austin, Tex. Alternatively, siRNAs can be chemically synthesized using ribonucleoside phosphoramidites and a DNA/RNA synthesizer. Sequences that the siRNA were designed to are as follows (sequence of sense strand shown): SOD1 (5′ thiol on sense): AAU CCU CAC UCU AAG AAA CAU (SEQ ID NO: 1)(GenBank Accession No. M25157, initiation at base 59, target bases 135-155); SOD1 (5′ thiol on antisense): AAC CAG UGG UGG UGU CAG GAC (SEQ ID NO: 2)(GenBank Accession No. NM017050, initiation at base 94, target bases 289-309); Casp8 (5′ thiol on antisense, 5′ FITC on sense): AAG CAC AGA GAG AAG AAU GAG (SEQ ID NO: 3)(GenBank Accession No. BC006737, initiation at base 336, target bases 878-898); Casp8 (5′ thiol on antisense): AAG AAG CAG GAG ACC AUC GAG (SEQ ID NO: 4)(GenBank Accession No. BC006737, initiation at base 336, target bases 432-452); and Casp9 (5′ thiol on antisense): AAG GCA CCC UGG CUU CAC UCU (SEQ ID NO: 5)(GenBank Accession No. NM015733, initiation at base 1, target bases 245-265).

### Example 2

Penetratin1 (mw 2503.93) (QBiogene, Inc., Carlsbad, Calif.) was reconstituted to 2 mg/ml in RNase/DNase sterile water (0.8 mM). siRNA (double-stranded, annealed, and synthesized with a 5′ -thiol group on the sense or antisense strand) was reconstituted to 88 μM in RNase-/DNase-free sterile water. To link the penetratin1 to the siRNA, 25 μl of penetratin1 were added to 25 μl of the diluted oligo, for total volume of 250 μl. This mixture was incubated for 15 min at 65° C., followed by 60 min at 37° C., then stored at 4° C. Alternatively, where only small amounts of the mixture are required, these may be aliquoted and stored at −80° C. Linkage can be checked by running the vector-linked siRNA and an aliquot that has been reduced with DTT on a 15% non-denaturing PAGE. siRNA can be visualized with SyBrGreen (Molecular Probes, Eugene, Oreg.).

### Example 3

Cell cultures used in Examples 5-9 were prepared as follows. Sympathetic neuron cultures were prepared from 1-day-old wild-type and caspase-2^{-/-} mouse pups (Bergeron et al., “Defects in regulation of apoptosis in caspase-2-deficient mice,” Genes Dev., 12, 1304-1314 (1998)), as previously described (Troy, et al., “Caspase-2 mediates neuronal cell death induced by beta-amyloid,” J. Neurosci., 20, 1386-1392 (2000)). Cultures were grown in 24-well collagen-coated dishes for survival experiments, and in 6-well collagen-coated dishes for RNA and protein extraction in RPMI 1640 medium (Omega Scientific, Tarzana, Calif.; ATCC, Manassas, Va.) plus 10% horse serum with mouse NGF (100 ng/ml). One day following plating, uridine and 5-fluorodeoxyuridine (10 μM each) were added to the cultures, and left for three days to eliminate non-neuronal cells. (Less than 1% non-neuronal cells remain after 3 days.)

Hippocampi were dissected from embryonic day 18 (E18) rat fetuses, dissociated by trituration in serum-free medium, plated on 0.1 mg/ml poly-D-lysine-coated tissue culture wells or plastic Lab-Tek slide wells, and maintained in a serum-free environment. The medium consisted of a 1:1 mixture of Eagle's MEM and Ham's F12 (Gibco, Gaithersburg, Md.) supplemented with glucose (6 mg/ml), putrescine (60 μM), progesterone (20 nM), transferrin (100 μg/ml), selenium (30 nM), penicillin (0.5 U/ml), and streptomycin (0.5 μg/ml) (Sigma, St. Louis, Mo.). In all experiments, neurons were cultured for 4-5 days before treatment. Cultures contained <2% glial cells, as confirmed by staining for glial markers.

### Example 4

Immunocytochemistry in Examples 5-9 was performed according to the following protocol. Cultured cells were fixed with 4% paraformaldehyde, exposed to primary antibodies at room temp for 1.5 h, washed with PBS, exposed to the appropriate fluorescent secondary antibodies for 1 h at room temperature, followed by Hoechst stain for 15 min at room temperature, and then analyzed with a Nikon fluorescent microscope. For uptake studies, living cultures were treated with FITC-siRNA, and analyzed with a Perkin-Elmer Spinning Disc confocal imaging system mounted on a Nikon inverted microscope.

### Example 5

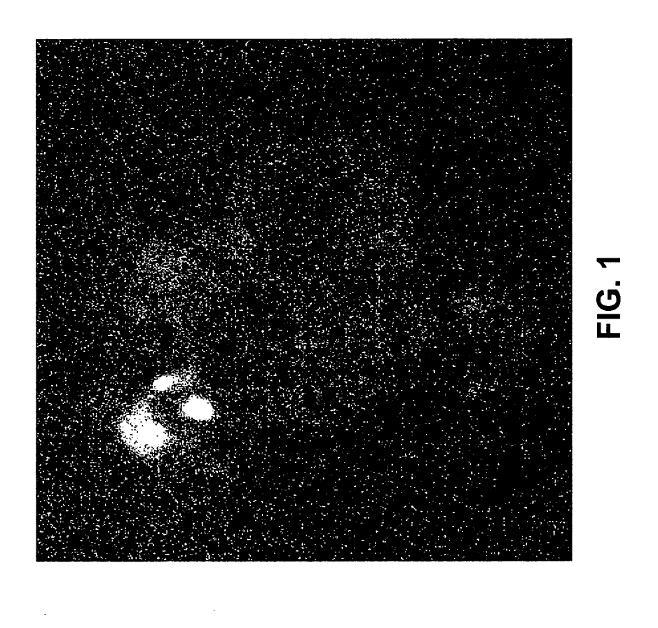

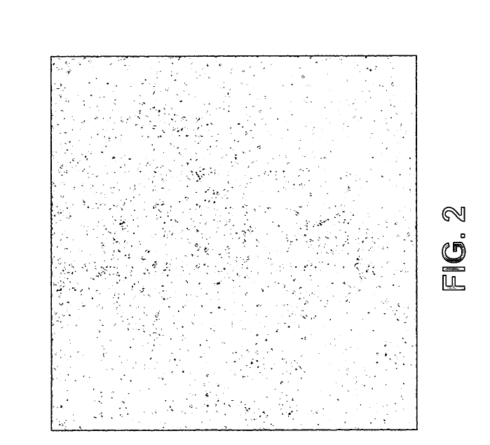

Transfection efficiencies of neuronal cells are generally low. To increase efficiency of delivery of siRNA to neuronal cells, the inventors designed small interfering ribonucleic acid molecules that could be linked to a cell-penetrating peptide. Specifically, either of the sense or antisense strand of each small interfering RNA was modified at its 5′ end with a thiol group, and covalently bonded via a disulfide bond with a penetratin 1 peptide having a pyridyl disulfide function at its terminal end. siRNA labeled with FITC was linked to the penetratin1 peptide, and applied to cultured rat sympathetic neurons as prepared in Example 3. FITC was visualized with confocal microscopy. Uptake was rapid, within minutes of application of siRNA, as shown in FIG. 1. Cultured hippocampal neurons were treated in the same way, and cultures were visualized two days later; the siRNA-FITC was still visible in the cytoplasm after two days, as shown in FIG. 2.

### Example 6

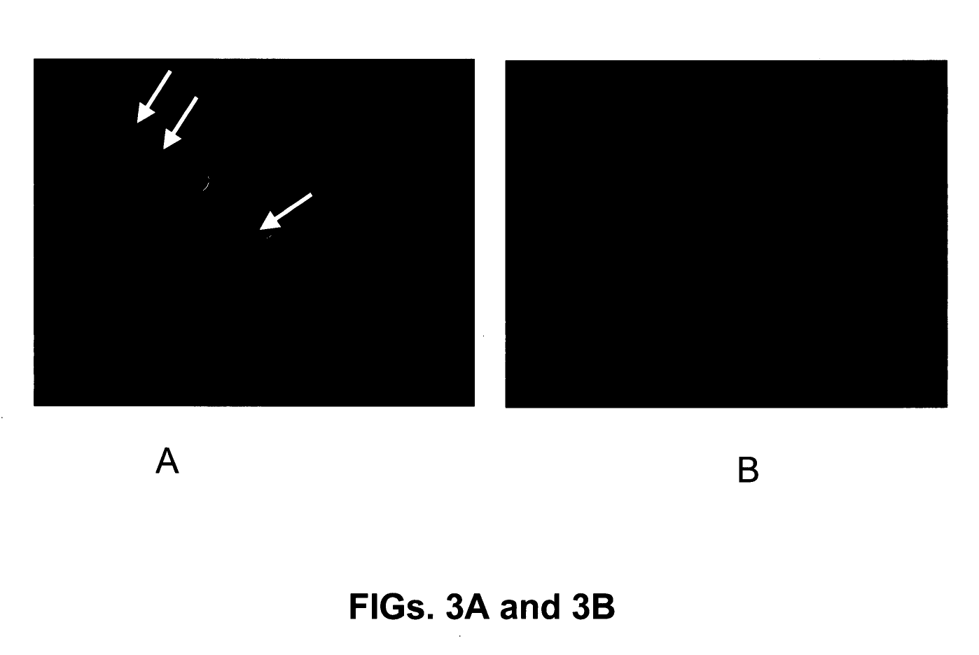

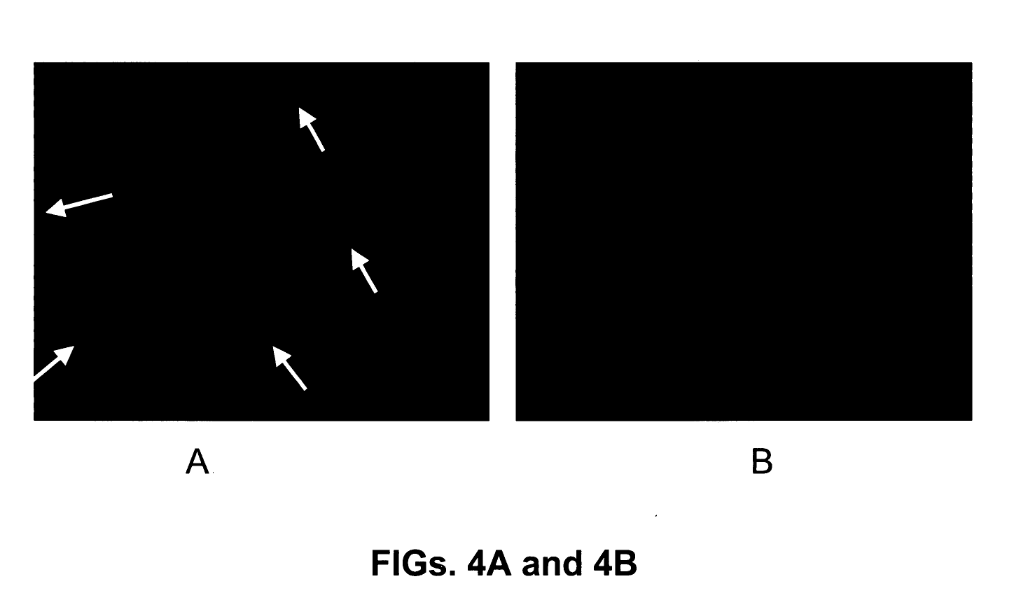

siRNA were designed for two members of the caspase family of death proteases, caspase-8 and caspase-9, and linked to the penetratin 1 peptide. Cultured mouse sympathetic neurons were treated with each of these constructs. Cultures were grown for one day, fixed and immunostained for caspase-8 or caspase-9, together with Hoechst stain, and then visualized with fluorescent microscopy (FIGS. 3 and 4). Expression of the targeted caspase (caspase-8 or caspase-9) was inhibited in all of the cultured cells. Expression of non-targeted caspases was not changed.

### Example 7

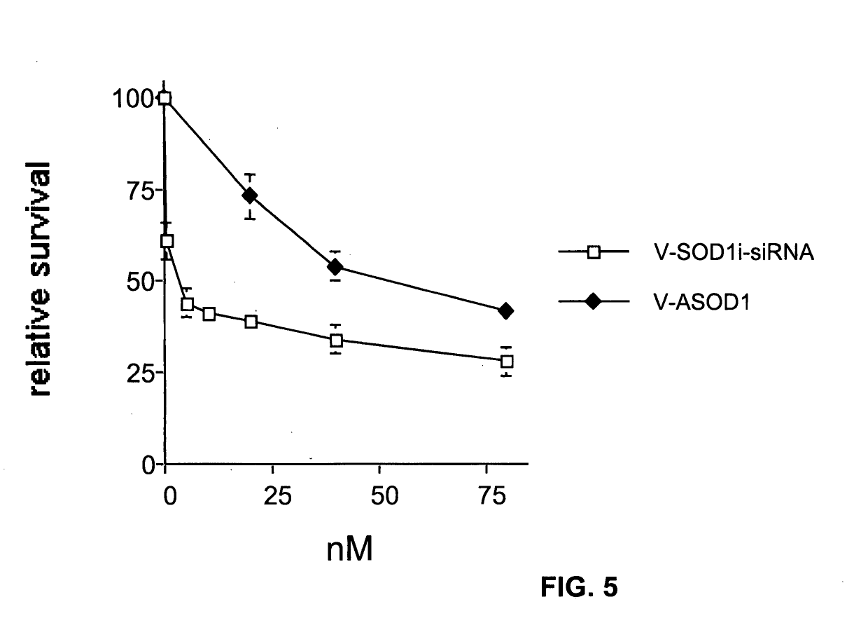

The inventors have previously shown that antisense oligonucleotides to SOD1 can downregulate SOD1-specific activity in a dose-dependent manner (Troy, et al., “Downregulation of Cu/Zn superoxide dismutase leads to cell death via the nitric oxide-peroxynitrite pathway,” J. Neurosci., 16, 253-261(1996)). Presently, the inventors determined that siRNA targeted to SOD1 could elicit the same effect. Cultured hippocampal neurons were treated with various concentrations of V-SOD1i (siRNA targeted to SOD1), and assayed for SOD activity. As shown in FIG. 5, there was a dose-dependent inhibition of SOD1-specific activity.

### Example 8

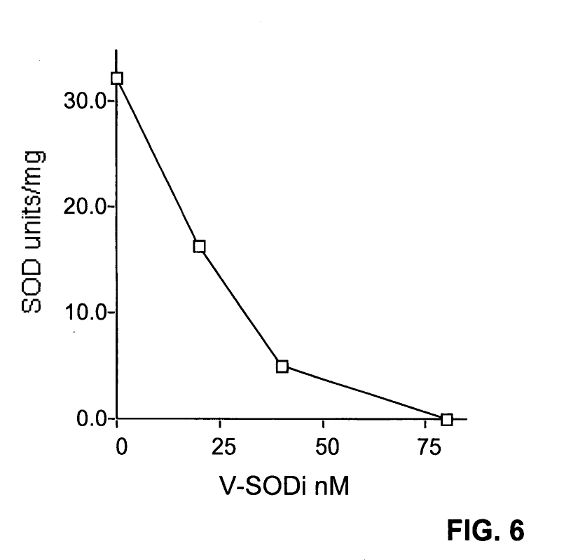

The inventors compared the efficacy of the siRNA and the antisense oligonucleotide in inducing death in cultured neurons. Relative survival of hippocampal neurons treated with either construct at the indicated concentrations was determined, as illustrated in FIG. 6. The siRNA was at least 10 times more potent in inducing death of the hippocampal neurons. siRNA unrelated to SOD1 did not affect the survival of hippocampal neurons.

### Example 9

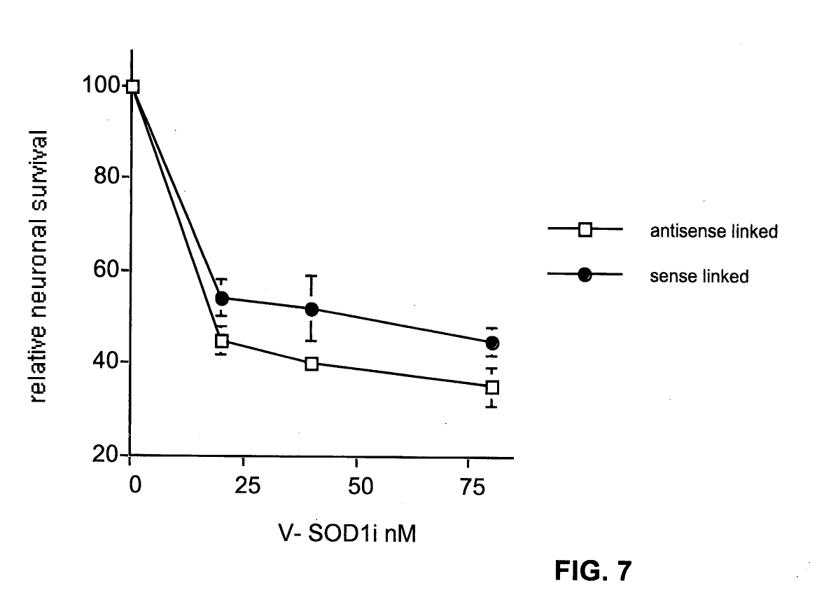

siRNA is double-stranded, and either the sense or the antisense strand can be modified with the thiol group at the 5′ end for linkage to the vector peptide. The inventors tested which strand was preferable using siRNA targeted to SOD 1, and determined survival of hippocampal neurons treated with the indicated concentrations of each construct (FIG. 7). The constructs were equally effective in inducing death, suggesting that the vector peptide can be linked to either strand.

All publications, patent applications and issued patents cited in this specification are herein incorporated by reference as if each individual publication, patent application or issued patent were specifically and individually indicated to be incorporated by reference. Further, the earlier incorporation by reference of any specific publication, patent application or issued patent shall not negate this paragraph. The citation of any publication, patent application or issued patent is for its disclosure prior to the filing date of the subject application and should not be construed as an admission that the present invention is not entitled to antedate such disclosure by virtue of prior invention.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A membrane-permeable complex for facilitating delivery of a double-stranded ribonucleic acid molecule into a cell, comprising a double-stranded ribonucleic acid molecule, a cell-penetrating peptide, and a covalent bond linking the double-stranded ribonucleic acid to the cell-penetrating peptide.

2. The membrane-permeable complex of claim 1, wherein at least one strand of the double-stranded ribonucleic acid is modified at its 5′ end for linkage with the cell-penetrating peptide, and the covalent bond links the 5′ modified strand to the cell-penetrating peptide.

3. The membrane-permeable complex of claim 1, wherein the double-stranded ribonucleic acid molecule is a small temporal RNA.

4. The membrane-permeable complex of claim 1, wherein the double-stranded ribonucleic acid is a short hairpin RNA.

5. The membrane-permeable complex of claim 1, wherein the double-stranded ribonucleic acid molecule is a microRNA.

6. The membrane-permeable complex of claim 1, wherein the double-stranded ribonucleic acid molecule is a small interfering RNA.

7. The membrane-permeable complex of claim 1, wherein at least one strand of the double-stranded ribonucleic acid molecule is homologous to a portion of mRNA transcribed from the human SOD1 gene.

8. The membrane-permeable complex of claim 7, wherein at least one strand of the double-stranded ribonucleic acid molecule comprises the nucleotide sequence of SEQ ID NO: 1.

9. The membrane-permeable complex of claim 7, wherein at least one strand of the double-stranded ribonucleic acid molecule comprises the nucleotide sequence of SEQ ID NO: 2.

10. The membrane-permeable complex of claim 1, wherein at least one strand of the double-stranded ribonucleic acid molecule is homologous to a portion of mRNA transcribed from the human caspase 8 gene.

11. The membrane-permeable complex of claim 10, wherein at least one strand of the double-stranded ribonucleic acid molecule comprises the nucleotide sequence of SEQ ID NO: 3.

12. The membrane-permeable complex of claim 10, wherein at least one strand of the double-stranded ribonucleic acid molecule comprises the nucleotide sequence of SEQ ID NO: 4.

13. The membrane-permeable complex of claim 1, wherein at least one strand of the double-stranded ribonucleic acid molecule is homologous to a portion of mRNA transcribed from the human caspase 9 gene.

14. The membrane-permeable complex of claim 13, wherein at least one strand of the double-stranded ribonucleic acid molecule comprises the nucleotide sequence of SEQ ID NO: 5.

15. The membrane-permeable complex of claim 2, wherein the 5′ end is modified with a thiol group.

16. The membrane-permeable complex of claim 15, wherein the covalent bond linking the 5′ modified end to the cell-penetrating peptide is a disulfide bond.

17. The membrane-permeable complex of claim 1, wherein the cell-penetrating peptide is selected from the group consisting of penetratin, transportan, pIsl, TAT, pVEC, MTS and MAP.

18. The membrane-permeable complex of claim 17, wherein the cell-penetrating peptide is penetratin.

19. The membrane-permeable complex of claim 1, further comprising a label affixed to at least one strand of the double-stranded ribonucleic acid molecule.

20. The membrane-permeable complex of claim 19, wherein the label is affixed to the 5′ end of at least one strand of the double-stranded ribonucleic acid molecule.

21. The membrane-permeable complex of claim 20, wherein the label is an enzyme label, a chemical label, or a radioactive label.

22. The membrane-permeable complex of claim 1, further comprising a moiety conferring target cell specificity to the membrane-permeable complex.

23. A composition, comprising the membrane-permeable complex of claim 1 and a pharmaceutically acceptable carrier, excipient or diluent.

24. A membrane-permeable complex for facilitating delivery of a small interfering RNA molecule into a cell, comprising:
(i) a small interfering RNA molecule comprising a duplex region of at least 19 nucleotides, wherein at least one strand of said duplex is homologous to a target mRNA in a cell, and wherein at least one strand of the small interfering RNA molecule is modified at its 5′ end for linkage with a cell penetrating peptide,
(ii) a cell-penetrating peptide selected from the group consisting of penetratin, transportan, pIsl, TAT, pVEC, MTS and MAP; and
(iii) a covalent bond linking the small interfering RNA molecule to the cell-penetrating peptide.

25. A method of facilitating delivery of a double-stranded ribonucleic acid molecule into a cell, comprising the steps of:
(a) providing a membrane-permeable complex, wherein said complex comprises a double-stranded ribonucleic acid molecule, a cell-penetrating peptide, and a covalent bond linking the double-stranded ribonucleic acid molecule to the cell-penetrating peptide; and
(b) contacting the cell with the membrane-permeable complex so that said complex is delivered into the cell.

26. The method of claim 25, wherein at least one strand of the double-stranded ribonucleic acid molecule is modified at its 5′ end for linkage with the cell-penetrating peptide, and the covalent bond links the 5′ modified strand to the cell-penetrating peptide.

27. The method of claim 25, wherein the double-stranded ribonucleic acid molecule is a small temporal RNA.

28. The method of claim 25, wherein the double-stranded ribonucleic acid is a short hairpin RNA.

29. The method of claim 25, wherein the double-stranded ribonucleic acid molecule is a microRNA.

30. The method of claim 25, wherein the double-stranded ribonucleic acid molecule is a small interfering RNA.

31. The method of claim 25, wherein the cell is in a tissue.

32. The method of claim 25, wherein the cell is mammalian.

33. The method of claim 25, wherein the cell is human.

34. The method of claim 25, wherein the membrane-permeable complex is delivered to the cell ex vivo.

35. The method of claim 25, wherein the membrane-permeable complex is delivered to the cell in vivo.

36. The method of claim 35, wherein the membrane-permeable complex is delivered to the cell in vivo via oral, parenteral, rectal, intradermal, transdermal or topical administration to a subject.

37. The method of claim 25, wherein at least one strand of the double-stranded ribonucleic acid molecule is homologous to a portion of mRNA transcribed from a target gene.

38. The method of claim 37, wherein the target gene is an endogenous gene.

39. The method of claim 37, wherein the target gene is a heterologous gene.

40. The method of claim 26, wherein the 5′ end is modified with a thiol group.

41. The method of claim 40, wherein the covalent bond linking the 5′ modified end to the cell-penetrating peptide is a disulfide bond.

42. The method of claim 25, wherein the cell-penetrating peptide is selected from the group consisting of penetratin, transportan, pIsl, TAT, pVEC, MTS and MAP.

43. The method of claim 42, wherein the cell-penetrating peptide is penetratin.

44. A method of facilitating delivery of a small interfering RNA molecule into a cell, comprising the steps of:
(a) providing a membrane-permeable complex, wherein said complex comprises:
(i) a small interfering RNA molecule comprising a duplex region of at least 19 nucleotides, wherein at least one strand of the small interfering RNA molecule is homologous to a target mRNA in a cell, and wherein at least one strand of the small interfering RNA molecule is modified at its 5′ end for linkage with a cell penetrating peptide;
(ii) a cell-penetrating peptide selected from the group consisting of penetratin, transportan, pIsl, TAT, pVEC, MTS and MAP; and
(iii) a covalent bond linking the small interfering RNA molecule to the cell-penetrating peptide; and
(b) contacting the cell with the membrane-permeable complex so that said complex is delivered into the cell.

45. A method of inhibiting expression of a target gene in a cell, comprising the steps of:
(a) providing a membrane-permeable complex for inhibiting expression of a target gene, wherein said complex comprises
(i) a double-stranded ribonucleic acid molecule, with at least one strand of said molecule having a nucleotide sequence which is homologous to a portion of mRNA transcribed from the target gene,
(ii) a cell-penetrating peptide, and
(iii) a covalent bond linking the double-stranded ribonucleic acid molecule to the cell-penetrating peptide; and
(b) contacting the cell to the membrane-permeable complex so that said complex is delivered into the cell in an amount sufficient to inhibit expression of the target gene.

46. The method of claim 45, wherein at least one strand of the double-stranded ribonucleic acid molecule of the~membrane-permeable complex is modified at its 5′ end for linkage with the cell-penetrating peptide, and the covalent bond links the 5′ modified strand to the cell-penetrating peptide.

47. The method of claim 45, wherein the double-stranded ribonucleic acid molecule is a small interfering RNA.

48. The method of claim 45, wherein the cell-penetrating peptide is selected from the group consisting of penetratin, transportan, pIsl, TAT, pVEC, MTS and MAP.

49. The method of claim 48, wherein the cell-penetrating peptide is penetratin.

50. The method of claim 45, wherein the cell is in a tissue.

51. The method of claim 50, wherein the cell is mammalian.

52. The method of claim 51, wherein the cell is human.

53. The method of claim 45, wherein the membrane-permeable complex is provided to the cell or tissue ex vivo.

54. The method of claim 45, wherein the membrane-permeable complex is provided to the cell or tissue in vivo.

55. The method of claim 54, wherein the membrane-permeable complex is provided to the cell in vivo via oral, parenteral, rectal, intradermal, transdermal or topical administration to a subject.

56. The method of claim 45, wherein the target gene is an endogenous gene.

57. The method of claim 56, wherein uninhibited expression of the target gene results in a disease or condition.

58. The method of claim 45, wherein the target gene is a heterologous gene.

60. The method of claim 58, wherein uninhibited expression of the target gene results in a disease or condition.

61. A method of determining the function of a target gene in a cell, comprising the steps of:
(a) providing a membrane-permeable complex for inhibiting expression of the target gene, wherein said complex comprises
(i) a double-stranded ribonucleic acid molecule, with at least one strand of said molecule having a nucleotide sequence which is homologous to a portion of mRNA transcribed from the target gene,
(ii) a cell-penetrating peptide; and
(iii) a covalent bond linking the double-stranded ribonucleic acid molecule to the cell-penetrating peptide;
(b) contacting the cell with the membrane-permeable complex so that said complex is delivered into the cell in an amount sufficient to inhibit expression of the target gene; and
(c) observing the phenotype of the cell resulting from step (b) to that of an appropriate control cell, thereby determining information regarding the function of the target gene in the cell.


SEQUENCE LISTING (12 sequences)

SEQ ID NO: 1 - RNA, 21 nt, Homo sapiens
  misc_RNA (1)..(21)
aauccucacucuaagaaacau

SEQ ID NO: 2 - RNA, 21 nt, Homo sapiens
aaccagugguggugucaggac

SEQ ID NO: 3 - RNA, 21 nt, Homo sapiens
  misc_RNA (1)..(21)
aagcacagagagaagaaugag

SEQ ID NO: 4 - RNA, 21 nt, Homo sapiens
  misc_RNA (1)..(21)
aagaagcaggagaccaucgag

SEQ ID NO: 5 - RNA, 21 nt, Mus musculis
  misc_RNA (1)..(21)
aaggcacccuggcuucacucu

SEQ ID NO: 6 - protein, 16 aa, Drosophila antennapedia
  PEPTIDE (1)..(16)
RQIKIWFQNRRMKWKK

SEQ ID NO: 7 - protein, 27 aa, Artificial sequence
  Transportan is a 27 amino acid-long peptide containing 12 functional amino acids from the amino terminus of the neuropeptide galan in and the 14 amino acid-long wasp venom peptide toxin,mastoparan, in the carboxyl terminus, connected via a lysine.
GWTLNSAGYLLGKINLKALAALAKKIL

SEQ ID NO: 8 - protein, 16 aa, Rattus sp.
  PEPTIDE (1)..(16)
PVIRVWFQNKRCKDKK

SEQ ID NO: 9 - protein, 13 aa, Human immunodeficiency virus type 1
GRKKRRQRRRPPQ

SEQ ID NO: 10 - protein, 16 aa, Mus musculis
  PEPTIDE (1)..(16)
LLIILRRRIRKQAHAH

SEQ ID NO: 11 - protein, 27 aa, Artificial sequence
  MPS peptide is a chimera of the hydrophobic terminal domain of the viral gp41 protein and the nuclear localization signal from simian virus 40 large antigen.
GALFLGWLGAAGSTMGAWSQPKKKRKV

SEQ ID NO: 12 - protein, 19 aa, Artificial sequence
  alpha-helical amphipathic model peptide
KLALKLALKALKAALKLAX